(19) United States
(12) Patent Application Publication
(10) Pub. No.: US 2014/0093891 A1
(43) Pub. Date: Apr. 3, 2014

(54) DEVICE FOR PERFORMING A BIOCHEMICAL ANALYSIS, ESPECIALLY IN OUTER SPACE

(71) Applicant: Astrium GmbH, Taufkirchen (DE)

(72) Inventors: Peter KERN, Salem (DE); Jessica JANSON, Wahlwinkel (DE); Crispin SZYDZIK, Napoleons VIC (AU)

(73) Assignee: Astrium GmbH, Taufkirchen (DE)

(21) Appl. No.: 14/028,790

(22) Filed: Sep. 17, 2013

(30) Foreign Application Priority Data
Oct. 1, 2012 (DE) 10 2012 109 317.2

(51) Int. Cl. G01N 33/53

(52) U.S. Cl. CPC G01N 33/5306
USPC 435/7.72; 436/501; 435/287.2; 422/69

(57) ABSTRACT

A device for performing a biochemical analysis, especially in outer space, more particularly an immunoassay, in which analysis at least one analyte in a sample is determined selectively, having at least one reaction container which has at least one work volume which is intended for taking in a liquid volume and for performing at least one substep of an analysis reaction, and having at least one interface which is intended for connecting at least one work volume to a further media container.

### PRIOR ART

The invention relates to a device according to the preamble of Claim 1.

A frequently used biochemical analytical technique for qualitatively and/or quantitatively detecting an analyte in a sample is provided by the methods referred to as immunoassays. Immunoassays are based on the functional principle of selective binding of an analyte in the sample by an analyte-specific pair of capture antibodies (cAB) and detection antibodies (dAB), with the latter bearing bound to itself a labeling substance or being intended for binding of the labeling substance over the course of the method. The capture antibodies are intended to fix the analyte on a solid location, for example a surface on which the capture antibodies are bound, or on carrier particles for the capture antibodies. The detection antibody binds selectively to the analyte or to the capture antibody. By means of the labeling substance, a measurable signal is produced which is intended to allow detection of a resulting analyte complex composed of analyte, capture antibody and detection antibody. In the immunoassays referred to as so-called enzyme-linked immunosorbent assays (ELISAs), the analyte is labeled by means of an enzyme as labeling substance, which is present fixed on the detection antibody or is bound to the detection antibody in a further reaction step, with a chromogenic or a luminescent compound, for example a chemiluminescent, electroluminescent, bioluminescent or fluorescent compound, being generated from an added substrate in a subsequent enzyme-catalyzed reaction, which compound can be detected using optical techniques. To avoid signal saturation of the chromogenic or luminescent compound, a stopper is added after a predefined period to interrupt the enzyme-catalyzed reaction. The stopper can cause the interruption by, for example, a change in pH, and by means of the pH change, a resulting product from the reaction of the substrate with the enzyme is frequently made visible in the manner of a pH indicator. In the case of so-called radioimmunoassays (RIAs), radioactive substances are used as labeling substances bound to the detection antibody, with the analyte being quantitatively determined via measurement of the radioactivity. Especially for precise, quantitative determination of the analyte, it is necessary to carefully mix the sample, the capture antibodies, the detection antibodies and the labeling substance. Under normal conditions, this mixing is achieved by combination of the individual constituents and subsequent mixing by means of movement of reaction vessels, for example by means of rotating mixers. Under normal conditions, excess substance amounts are removed by simple pouring. Especially under conditions of reduced gravity, for example in the case of experiments in outer space, removal of excess substance amounts by the force of gravity is not available. Moreover, mixing under conditions of reduced gravity must be carried out in such a way that other experiments in close proximity are not disturbed, for example because of vibrations.

### ADVANTAGES OF THE INVENTION

The invention is based on a device for performing a biochemical analysis, especially in outer space, more particularly an immunoassay, in which analysis at least one analyte in a sample is determined selectively, having at least one reaction container which has at least one work volume which is intended for taking in a liquid volume and for performing at least one substep of an analysis reaction, and having at least one interface which is intended for connecting at least one work volume to a further media container. In the work volume, substances for performing the reaction can be already stored, in a bound or in an unbound state, prior to starting analysis, more particularly prior to adding the sample. In principle, instead of liquid volumes, it is also possible to take in gas volumes in the work volume, for example by introducing a gas for displacement of a liquid volume in a substep of an analysis. “Stored bound” is to be understood to mean in particular bound to a surface of the work volume, wherein a substance stored bound can be detached over the course of a reaction process and brought into solution. Furthermore, “stored bound” is to be understood to mean that substances are irreversibly bound or fixed on solid geometric sites in the work volume. “Performance in outer space” is to be understood to mean in particular that the biochemical analysis is performed beyond Earth, for example in a spacecraft in Earth orbit or at a Lagrange point, during a spaceflight or an orbit around another planet or a moon, on a satellite, a moon, an asteroid or on a planet other than Earth. More particularly, the performance in outer space can take place under conditions of reduced gravity. “Conditions of reduced gravity” are to be understood to mean in particular conditions in which a gravity effect of maximally 0.9 g, advantageously maximally 1*10⁻³ g, preferably maximally 1*10⁻⁶ g and particularly preferably maximally 1*10⁻⁸ g is effective. The gravity effect can be generated by gravitation and/or artificially by acceleration. The value of 9.81 m/s² for acceleration due to gravity on Earth is designated “g”. An “interface” is to be understood to mean in particular an element which is intended to establish a completely closed connection between the work volume and the further media container. A “completely closed connection” is to be understood to mean in particular that media flow via the connection is completely isolated from an external environment by the interface and, more particularly, substance escape into the external environment is prevented. For example, the interface can be designed to form a connection with the further media container according to the Luer-Lock principle or the interface can have septa, with substance passage through the septa being achieved by means of penetration or displacement.

It is proposed that the reaction container be implemented as a container which is at least substantially completely closed in the assembled state. “At least substantially completely closed” is to be understood to mean in particular that the vessel, at least in an assembled state for performing a biochemical analysis, is free of openings except for coupling openings which are intended for coupling to further vessels for taking in reaction starting materials or reaction products, and so an escape of reaction starting materials and/or products is prevented. “At least substantially completely closed in the assembled state” is to be understood to mean in particular that the reaction container is designed such that a connection to a further element, for example a commercially available planar array support for capture antibodies or a commercially available multiwell plate, is intended for complete closure of the reaction container. It is possible in particular to achieve high process safety and universal usability for analysis of hazardous substances, for example acidic, basic or toxic substances, and under extreme conditions, for example conditions of reduced gravity, especially in outer space.

It is further proposed that the work volume be designed for reaction performance under conditions of reduced gravity. More particularly, under conditions of reduced gravity, behavior of liquids is dominated by surface tension and the work volume has a design specifically adapted to said behavior. It is possible in particular to achieve a device which makes it possible to perform a reaction reproducibly and in a controlled manner with reduced gravity-based interference factors.

It is further proposed that the work volume have a shape which widens starting from an interface. A “shape which widens starting from an interface” is to be understood to mean in particular that the work volume has a shape in which, viewed in a plane in which a longitudinal extent of the interface passes and in which an inflow vector of liquid volumes is situated, proceeding from the interface, there is monotonic enlargement of a diameter of the work volume transverse to an outflow direction from the interface up to a site of maximum extension of the diameter of the work volume transverse to the outflow direction. After a site of maximum extension, the diameter of the work volume transverse to the outflow direction can decrease in particular in the outflow direction. In particular, when introducing liquid volumes into the work volume, a rapid enlargement of a surface covered by the liquid volume is thus attained. Under conditions of reduced gravity, when introducing a new liquid volume into a volume at least partly filled by a further liquid volume, it is possible in particular to achieve displacement of the further liquid volume by the new liquid volume, since diffusive mixing is low under reduced gravity and, owing to the shape of the work volume, introduction of the new liquid volume does not result in any residual volumes of the further liquid volume remaining behind a front of the new liquid volume. It is possible in particular to achieve a work volume in which, especially under conditions of reduced gravity, a change of liquid volumes is achieved with low losses due to mixing and/or with minimization of a required inflowing volume when changing liquid volumes in the work volume.

It is further proposed that the work volume be at least substantially rectangular. “At least substantially rectangular” is to be understood to mean in particular that the work volume, viewed in at least one plane, preferably in one plane, in which an inflow vector of liquid volumes is situated, has a rectangular shape, preferably a square shape, it being possible for one or more corners of the work volume to be rounded. It is possible in particular to achieve a work volume in which, especially under conditions of reduced gravity, a change of liquid volumes is achieved with low losses due to mixing and/or with minimization of a required inflowing volume when changing liquid volumes in the work volume.

It is further proposed that the work volume be in a drop shape. A drop shape is to be understood to mean in particular a shape which has at least one entry opening and at least one exit opening and in which the work volume, viewed in at least one plane, preferably in one plane, in which an inflow vector of liquid volumes is situated, broadens in at least one subregion toward the exit opening. It is possible in particular to achieve a work volume in which, especially under conditions of reduced gravity, a change of liquid volumes is achieved with low losses due to mixing and/or with minimization of a required inflowing volume when changing liquid volumes in the work volume.

It is further proposed that the work volume be in a circular shape. It is possible in particular to achieve a work volume in which, especially under conditions of reduced gravity, a change of liquid volumes is achieved with low losses due to mixing and/or with minimization of a required inflowing volume when changing liquid volumes in the work volume.

It is further proposed that the work volume be in a nozzle shape. A “nozzle shape” is to be understood to mean in particular a shape which has at least one entry opening and at least one exit opening and in which the work volume tapers off in at least one subregion toward the exit opening. Preferably, the nozzle shape has at least two entry openings. It is possible in particular to achieve a work volume in which, especially under conditions of reduced gravity, a change of liquid volumes is achieved with low losses due to mixing and/or with minimization of a required inflowing volume when changing liquid volumes in the work volume.

Further proposed is at least one further media container implemented as a waste container which is intended for taking in excess liquid volumes. “Excess liquid volumes” is to be understood to mean in particular liquid volumes which are no longer required after a substep of the biochemical analysis, for example sample volumes containing unbound analytes after performance of a substep in which binding of the analyte to stationary capture antibodies is intended or liquid volumes containing unreacted detection antibodies. Preferably, the waste container is connected to at least one work volume via a connection by means of an interface. A small work volume and a compact device can be achieved in particular.

It is further proposed that the waste container be at least substantially completely closed. It is possible in particular to achieve high process safety and universal usability of the device for analysis of samples containing hazardous substances, for example acidic, basic or toxic substances, and/or for analysis taking place under extreme conditions.

It is further proposed that the waste container be designed for pressure-equalization operation. “Pressure-equalization operation” is to be understood to mean in particular that the waste container has at least one filter for pressure equalization with an environment, and so pressure buildup within the waste container and/or a pressure difference with respect to an environment can be avoided when introducing excess liquid volumes, more particularly under conditions of reduced gravity. Depending on media used in the analysis, the filter is implemented as a hydrophobic or hydrophilic filter. A waste container having increased safety for operation can be achieved in particular.

It is further proposed that the waste container have at least one wicking body. A “wicking body” is to be understood to mean in particular a capillary material which is intended to at least partly line the waste container on inner walls starting from an inlet and to take in and/or to transfer inflowing liquid volumes. The wicking body can be intended in particular for taking in excess liquid volumes and/or for storing absorbent material. An “absorbent material” is to be understood to mean in particular a material which is intended for taking in and for binding liquid volumes, for example organic absorbents, mineral adsorbents, sintered plastic storers, activated carbon or silica gel. More particularly, the wicking body is intended for taking in excess liquid volumes entering the waste container and for distributing them by means of the absorbent material for improved and speeded-up uptake and for preferably preventing re-escape of liquid volumes taken in. Preferably, the wicking body is further intended, for the purposes of attaining pressureless operation, for conducting gas present in the absorbent material during uptake of excess liquid volumes to an absorbent material-free region of the waste container, from which the gas can be released by means of a filter to achieve pressure equalization. It is possible in particular to achieve a waste container having rapid and safe uptake of excess liquid volumes and storage of the excess liquid volumes with high safety.

Further proposed is at least one further media container implemented as an analysis-material container which is intended for providing analysis materials. “Analysis materials” are to be understood to mean in particular materials necessary for the analysis reaction, for example capture and labeling antibodies and labeling substances of an immunoassay which are used for selectively determining the analyte, and also auxiliaries such as solvents and the like. Preferably, the analysis materials are stored in the analysis-material container in a required volumetric amount prior to the start of a method, and so only one release of the analysis materials has to be done for performance of the method. Alternatively, it is also possible to store the sample in the analysis-material container and to dispense with a separate sample container. An operationally and volumetrically reliable addition of the analysis materials can be achieved in particular.

It is further proposed that the analysis-material container be implemented as a multichamber syringe. A “multichamber syringe” is to be understood to mean in particular a container having a plurality of subchambers partitioned off by separators for separate storage of different reaction materials. Preferably, the multichamber syringe is designed to release the different analysis materials sequentially one after another, it being possible within the multichamber syringe to carry out controlled mixing of separately stored substances to give a substance mixture prior to release. Preferably, the multichamber syringe stores the required analysis materials in a substance amount specifically tailored to the analysis. In principle, storage of components of the analysis materials in, in each case, a separate analysis-material container can be carried out instead of using a multichamber syringe. It is possible in particular to reduce the number of analysis-material containers and to avoid errors in performing an analysis owing to absent analysis materials and/or analysis materials added in an insufficient amount.

It is further proposed that the analysis-material container be integrated with a waste container. “Be integrated” is to be understood to mean in particular that the analysis-material container has at least one compartment which is intended for taking in excess liquid volumes and which is preferably intended for taking in excess liquid volumes over the course of an analysis reaction in parallel to emptying of analysis-material storing compartments and for enlarging an uptake volume of said compartment during the uptake. The compartment can, for example, be implemented as a chamber of the analysis material container with a fixed or, preferably, with an alterable uptake volume, for example in the form of an elastic uptake sack or in the form of a chamber which is closed with a movable element. It is possible in particular to dispense with an additional, separate waste container and to reduce the system volume required.

It is further proposed that at least one reaction container be preassembled together with at least one further reaction container and/or at least one further media container to form a module which is intended for connection to a further media container. Preferably, the module has a waste container and an analysis-material container in addition to the reaction container, and so only the sample container needs to be connected via an interface for performance of the biochemical analysis. Preferably, the reaction container and the analysis-material container are already filled with analysis materials, and so it is possible to dispense with a filling step prior to performance of a biochemical analysis. It is possible in particular to achieve time savings in the performance of the biochemical analysis owing to preassembly of work units.

It is further proposed that the module be intended for allowing parallel performance of a plurality of biochemical analyses. More particularly, the module has for this purpose a plurality of reaction containers and/or a reaction container having a plurality of work volumes. More particularly, the module has for this purpose a configuration in which a plurality of reaction containers and/or work volumes are arranged in parallel. Savings in time and space can be achieved in particular.

Further proposed are magnetic mixing bodies which are intended for mixing reaction materials and the sample for the analysis reaction. “Magnetic mixing bodies” are to be understood to mean in particular magnetic and/or magnetizable bodies which are intended to be moved by means of an applied magnetic field, preferably an applied alternating magnetic field, for the purposes of mixing the reaction materials. Reliable mixing of the reaction materials can be achieved in particular.

Further proposed is a method for performing a biochemical analysis using a device according to the invention, in which method the performance is carried out under conditions of reduced gravity. More particularly, the method is designed in such a way that all substeps of the performance can be performed independently of the presence of gravity. Avoidance of gravity-based or mechanically caused disrupting influences can be achieved in particular.

It is further proposed that mixing of analysis materials and the sample for the analysis reaction be carried out by means of magnetic mixing bodies. Complete and efficient mixing, more particularly under conditions of reduced gravity in outer space, can be achieved in particular.

It is further proposed that only analysis materials and samples within a work volume of a reaction container be involved in an analysis reaction. More particularly, it is possible to dispense with volumetrically highly accurate provision of required volumes of analysis materials and/or samples and, instead, to add analysis materials and/or samples until the work volume, which defines a volume of participating substances, is filled. It is possible in particular to achieve a method which is easily performable and which is easily performable especially under conditions of reduced gravity.

It is further proposed that addition of analysis materials and samples can proceed in any desired small subvolumes and with pauses included. A flexibly adaptable method can be achieved in particular.

The device according to the invention is not to be restricted here to the above-described use and embodiment. More particularly, in order to fulfill a functionality described herein, the device according to the invention can have a number of individual elements, components and units differing from a number that is mentioned herein.

### DRAWINGS

Further advantages are revealed by the following description of the drawings. The drawings show 24 exemplary embodiments of the invention. The drawings, the description and the claims contain numerous features in combination. A person skilled in the art will appropriately also consider the features individually and combine them to form further meaningful combinations.

Shown by:

FIG. 1 is a diagram showing a device according to the invention having two reaction containers, each having a work volume, an analysis-material container, a waste container and a sample container,

FIG. 2 is a detailed view of a reaction container according to the invention,

FIG. 3 is a diagram showing a work volume in a circular shape,

FIG. 4 is a diagram showing an alternative work volume in a rectangular shape,

FIG. 5 is a diagram showing an alternative work volume in a drop shape,

FIG. 6 is a diagram showing an alternative work volume in a nozzle shape, having two inlets and one outlet,

FIGS. 7A, 7B, 7C, 7D, 7E are diagrams showing sequential process steps of a biochemical analysis in a and device according to the invention,

FIG. 8 is individual parts of a reaction container from FIG. 2 prior to assembly in a lateral view,

FIG. 9 is the reaction container from FIG. 2 in a partly assembled state in a diagrammatic lateral view,

FIG. 10 is the reaction container from FIG. 2 in an assembled state in a diagrammatic lateral view,

FIG. 11 is an alternative embodiment of a reaction container having a variable work volume in a diagrammatic lateral view,

FIG. 12 is an alternative embodiment of a reaction container having a variable work volume in a diagrammatic lateral view,

FIG. 13 is an embodiment of a reaction container having an alternative interface arrangement in a diagrammatic lateral view,

FIG. 14 is an embodiment of a reaction container having an alternative interface arrangement in a diagrammatic lateral view,

FIG. 15 is an embodiment of a reaction container having an alternative interface arrangement in a diagrammatic lateral view,

FIG. 16 is an embodiment of a reaction container having an alternative interface arrangement in a diagrammatic lateral view,

FIG. 17 is an alternative provision of capture antibodies in a reaction container according to FIG. 2, bound to magnet carrier bodies and in a solution,

FIG. 18 is an alternative provision of capture antibodies in a reaction container according to FIG. 2, which are present fixed or dried on a separate support material,

FIG. 19 is an alternative provision of capture antibodies in a reaction container according to FIG. 2 implemented as dried-in dots which are detached during reaction performance,

FIG. 20 is a diagram showing a waste container from FIG. 1 during a filling operation,

FIG. 21 is a diagram showing an alternative waste container during a filling operation,

FIG. 22 is a diagram showing an alternative waste container prior to a filling operation,

FIG. 23 is a diagram showing an alternative waste container during a filling operation,

FIG. 24 is a diagram showing an alternative waste container,

FIG. 25 is an alternative device having a reaction container which has two work volumes,

FIG. 26 is an alternative device in which reaction container, analysis-material container and waste container are preassembled to form a module,

FIG. 27 is an alternative device for parallel performance of a plurality of biochemical analyses,

FIG. 28A is an alternative device for parallel performance of a plurality of biochemical analyses, in which device a plurality of reaction containers are preassembled to form a module,

FIG. 28B is an alternative configuration of the device shown in FIG. 28A,

FIG. 29 is an alternative device in which a module composed of a plurality of reaction containers is charged successively by a multiport valve by means of elevated pressure,

FIG. 30 is an alternative device in which a module composed of a plurality of reaction containers is charged successively by a multiport valve by means of reduced pressure,

FIG. 31 is an alternative device for parallel performance of a plurality of biochemical analyses, in which device a plurality of reaction containers are preassembled to form a module,

FIG. 32 is an alternative device in which the reaction container is completely closed by connection to a commercial multiwell plate,

FIG. 33 is an alternative device in which the reaction container is completely closed by connection to a commercial planar array, and

FIG. 34 is an alternative device having an analysis-material container which is integrated with a waste container.

### DESCRIPTION OF THE EXEMPLARY EMBODIMENTS

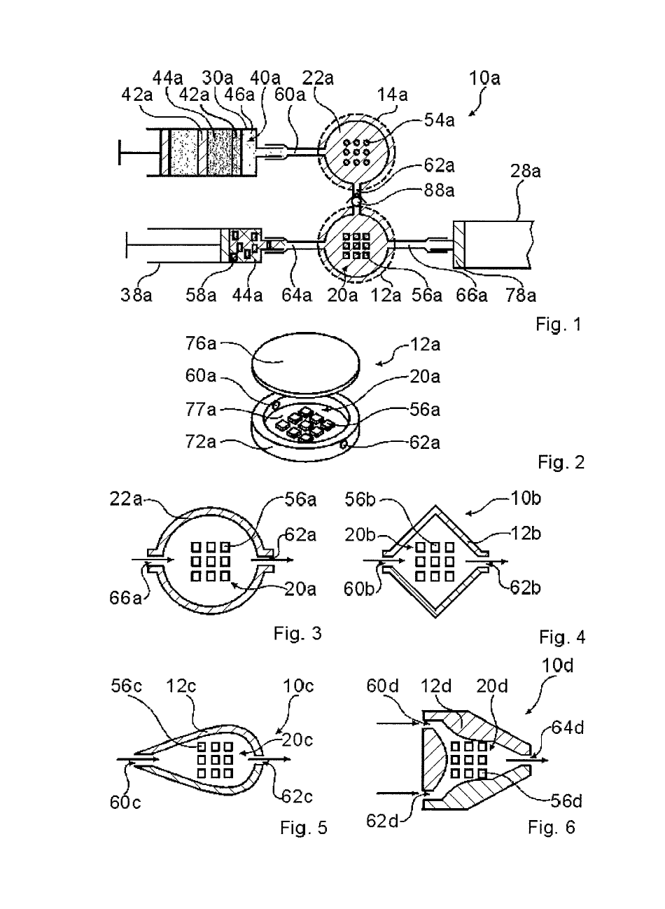

FIG. 1 shows a top view of a device 10a according to the invention for performing a biochemical analysis, formed by an immunoassay, in outer space, in which analysis an analyte in a sample 44a is determined selectively, having two reaction containers 12a, 14a which have in each case a work volume 20a, 22a which are intended for taking in a liquid volume and for performing at least one substep of an analysis reaction, and having four interfaces 60a, 62a, 64a, 66a which are intended for connecting at least the two work volumes 20a, 22a to one another and to three further media containers 28a, 30a, 38a. The interface 62a between the work volumes 20a, 22a has a valve 88a for preventing backflow from the work volume 20a into the work volume 22a. The reaction containers 12a, 14a are implemented as vessels which are substantially completely closed in the assembled state and which are only accessible via the interfaces 60a, 62a, 64a, 66a. The work volumes 20a, 22a are designed for reaction performance under conditions of reduced gravity and have a design specifically adapted to the behavior of liquids that is dominated by surface tension under conditions of reduced gravity. The work volumes 20a, 22a have a shape which widens starting from an interface 60a, 62a and 62, 64a, 66a, respectively. The work volumes 20a, 22a are in a circular shape. In work volume 20a of the reaction container 12a, capture antibodies 56a for the immunoassay are already bound to a surface of the work volume 20a prior to starting the immunoassay, and in the work volume 22a of the reaction container 14a, detection antibodies 54a in dried form are already present prior to starting the immunoassay and are brought into solution over the course of the immunoassay.

The device 10a further comprises a further media container implemented as a waste container 28a which is intended for taking in excess liquid volumes. Over the course of the method, the waste container 28a takes in liquid volumes which are no longer required, for example sample remnants with analyte which is unreacted and not bound to capture antibody 56a, and is connected to the work volume 20a via an interface 66a. The waste container 28a has a plunger 78a for enlarging an uptake volume and is substantially completely closed. The device 10a has in addition a further media container implemented as a sample container 38a which is connected to the work volume 20a via the interface 64a and is intended for feeding the sample 44a. The sample container 38a stores not only the sample 44a but also magnetic mixing bodies 58a which are intended for mixing analysis materials 46a, 48a, 50a, 52a and the sample 44a for an analysis reaction. The sample container 38a is substantially completely closed. The device 10a has in addition a further media container which is substantially completely closed and which is implemented as an analysis-material container 30a which is intended for providing analysis materials 46a, 48a, 50a, 52a. The analysis-material container 30a is implemented as a multichamber syringe having a plurality of subchambers 40a which are partitioned off by separators 42a and which are intended for separate storage of different analysis materials 46a, 48a, 50a, 52a. Connection of the analysis-material container 30a to the work volume 22a is achieved via the interface 60a. The analysis-material container 30a is in addition designed for sequential release of separately stored analysis materials 46a, 48a, 50a, 52a. Prior to transport of the device 10a into outer space, the analysis-material container 30a has been filled with the analysis materials 46a, 48a, 50a, 52a which are required for performing the biochemical analysis.

FIG. 2 shows the reaction container 12a having the work volume 20a in a more precise view in a partly assembled state. The reaction container 12a is composed of a base body 72a, a base 74a and a lid 76a to be placed thereon. One material of the reaction container 12a is formed by a transparent cyclic olefin copolymer, which has a low nonspecific binding capacity and allows evaluation of the immunoassay by means of optical techniques owing to transparency. In principle, the reaction container 12a can also be composed of another material, for example so-called “low-binding” polystyrene, the material being formed advantageously by a plastic and preferably by a transparent plastic.

FIG. 3 shows a diagram of the work volume 20a, which is in a circular shape with opposing interfaces 60a, 62a.

FIGS. 4-34 show, in addition to further details of the first exemplary embodiment of the invention, twenty-three further exemplary embodiments of the invention. The descriptions which follow and the drawings are essentially limited to the differences between the exemplary embodiments, and with regard to similarly designated components, especially with respect to components having the same reference signs, reference is made in principle also to the drawings and/or the description of FIG. 1. For the purposes of distinguishing the exemplary embodiments, the letter a is placed after the reference signs of the first exemplary embodiment in FIGS. 1-3. In the further exemplary embodiments of FIGS. 4 to 34, the letter a is replaced by the letters b to w. In FIGS. 4 to 34, the letter a is retained in the further exemplary embodiments in figure descriptions referring to the first exemplary embodiment.

FIG. 4 shows a section of an alternative device 10b having a reaction container 12b having a work volume 20b which is rectangular. Capture antibodies 56b are tightly bound in the work volume 20b. Interfaces 60b, 62b are arranged in two opposing corners of the rectangular shape. In principle, the interfaces 60b, 62b can also be arranged on adjacent corners of the rectangular shape or at least one of the interfaces 60b, 62b can be arranged in a wall region between corners, on the base or lid. The work volume 20b likewise has a shape which widens starting from an interface 60b, 62b. In alternative configurations of the device 10b, one or more of the corners of the rectangular work volume 20b, preferably corners away from the interfaces 60b, 62b, can be rounded.

An alternative device 10c has a reaction container 12c (FIG. 5) having a work volume 20c, which container is in a drop shape. An interface 60c is arranged on a pointy site of an edge of the drop shape, and an interface 62c is arranged on a site opposing the pointy site. Proceeding from the interface 60c, the work volume 20c widens, achieving adherence of inflowing liquids to walls of the work volume because of surface tension and, under conditions of reduced gravity, a change of liquid volumes with low losses due to mixing and with a reduced volume of a following medium.

An alternative device 10d comprises a reaction container 12d (FIG. 6) having a work volume 20d which is in a nozzle shape. The work volume 20d has two interfaces 60d, 62d on one side and also an interface 64d on a side opposing the two interfaces 60d, 62d. Via the two interfaces 60d, 62d, two different substance inflows can be provided at the same time or one after the other, making it possible to shorten process time and avoid substance losses through exchanging a media container.

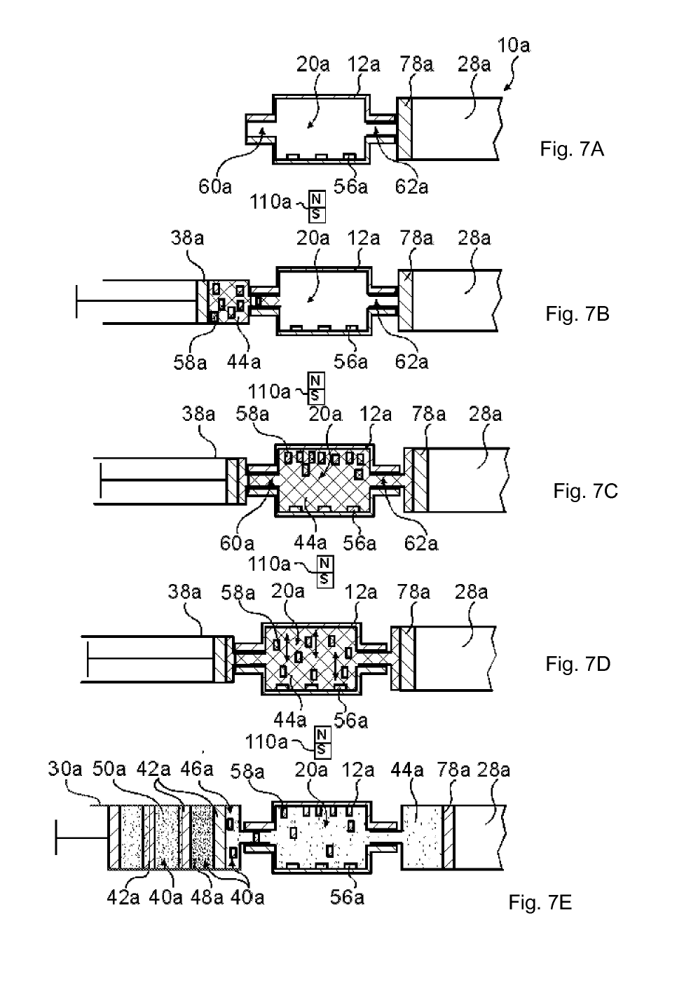

FIGS. 7A-7E show an exemplary depiction of a method for performing a biochemical analysis in the device 10a. The performance takes place under conditions of reduced gravity on board a spacecraft in outer space. In principle, the method can also be performed on an asteroid, a moon or even on Earth. Mixing of analysis materials 46a, 48a, 50a, 52a and the sample 44a for the analysis reaction is achieved by means of magnetic mixing bodies 58a. In a first method step (FIG. 7A), the reaction container 12a is only filled with the bound capture antibodies 56a and connected to the waste container 28a via the interface 62a. In a further method step (FIG. 7B), the sample container 38a is connected to the work volume 20a via the interface 60a. In a following method step (FIG. 7C), pressure is exerted on a movable plunger in the sample container 38a and, owing to the pressure, material of the sample 44a with the magnetic mixing bodies 58a is moved into the work volume 20a. In a following method step (FIG. 7D), mixing of the sample 44a is brought about by means of the magnetic mixing bodies 58a, which are set in motion via a magnet unit 110a, and as a result an analyte present in the sample 44a is brought past positions of the capture antibodies 56a and binds thereto. During filling, excess volume of the sample 44a is moved via the interface 62a from the work volume 20a of the reaction container 12a into the waste container 28a. In a following reaction step (FIG. 7E), the sample container 38a is replaced by the analysis-material container 30a. Via pressure on a moveable plunger of the analysis-material container 30a, the analysis-material container 30a is emptied analogously to emptying of the sample container 38a and analysis materials 46a, 48a, 50a, 52a are introduced successively into the work volume 20a. For example, the analysis material 46a can be formed by a neutral rinse solution which is used to displace liquid volumes containing unbound analyte from the work volume 20a and to move them into the waste container 28a. The analysis material 48a can then, for example, be formed by a solution containing detection antibodies 54a with labeling material bound thereto, which, as enzyme, is designed for cleavage of a substrate for signal production. The analysis material 48a is mixed by means of the magnet unit 110a and the magnetic mixing bodies 58a and detection antibodies 54a containing labels bind to the analytes bound to the capture antibodies 56a. In a further, exemplary step, a further rinse solution 52 is used to remove unbound detection antibodies 54a from the work volume. In an actual detection step of the exemplary method, in order to generate a detection signal using the analysis material 52a, substrate for cleavage by the labels is added, which substrate generates, for example, a fluorescent signal after cleavage. During the method, excess liquid volumes are transferred into the waste container 28a. In the method, an analysis reaction involves only analysis materials 46a, 48a, 50a, 52a and the sample 44a within the work volume 20a of the reaction container 12a, making it possible to dispense with volumetrically highly accurate measurement of analysis materials 46a, 48a, 50a, 52a and the sample 44a. Over the course of the method, addition of analysis materials 46a, 48a, 50a, 52a and the sample 44a can proceed in any desired small subvolumes and with pauses. In alternative method proceedings, the capture antibodies 56a can, for example, be added bound to magnetic carrier bodies instead of being bound to the base 74a. Furthermore, in alternative method proceedings, it is possible to use separate single-material containers for each of the analysis materials 46a, 48a, 50a, 52a instead of the analysis-material container 30a implemented as a multichamber syringe. In principle, other biochemical analysis methods can also be performed in the device 10a instead of immunoassays. When performed, the individual substeps of the method are not dependent on the presence of gravity and can thus be performed under conditions of reduced gravity. However, in principle, performance under normal gravity conditions on Earth is also possible.

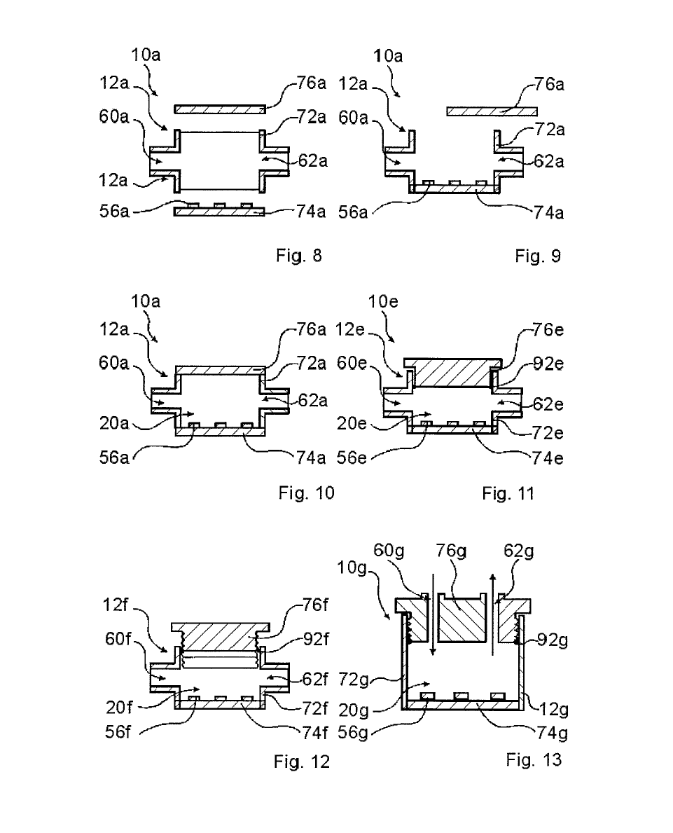

FIGS. 8-10 show an assembly operation for the reaction container 12a. In one assembly step (FIG. 8), the reaction container 12a is disassembled into individual parts formed by the lid 76a, the base body 72a with the interfaces 60a and 62a, and the base 74a with capture antibodies 56a bound thereto. In a following assembly step (FIG. 9), the base 74a is inserted into the base body 72a and attached securely by means of an adhesive operation. In a last assembly step (FIG. 10), the lid 76a is positioned in place and likewise attached using an glueing operation. Alternatively, instead of an glueing operation, it is also possible to undertake a different attachment operation, for example a welding process or a force-fit and/or interlock attachment of the lid 76a in the base body 72a.

FIG. 11 shows an alternative reaction container 12e of an alternative device 10e, which container comprises a base body 72e with interfaces 60e, 62e on lateral regions and a base 74e with capture antibodies 56e bound thereto. A lid 76e is pressed into the base body 72e and sealed using an O-ring 92e. Base body 72e, base 74e and lid 76e delimit a circular work volume 20e. By varying the depth at which the lid 76e is pressed in, it is possible to adjust the work volume 20e.

A further alternative device 10f (FIG. 12) has a reaction container 12f having a lid 76f which is screwable into a base body 72f having lateral interfaces 60f, 62f. The lid 76f can be screwed in at different depths, making it possible to vary a work volume 20f of the reaction container, and is sealed with an O-ring 92f. Capture antibodies 56f are bound to a base 74f.

FIG. 13 shows an alternative reaction container 12g of an alternative device 10g, which container is substantially similar to the previous exemplary embodiment, having a work volume 20g. A lid 76g screwed into a base body 72g is sealed with an O-ring 92g and has two interfaces 60g, 62g which are intended for supply and discharge of liquid volumes. Capture antibodies 56g for performance of an immunoassay are bound to a base 74g. Alternatively, it is also possible for detection antibodies or other required analysis materials to be bound to the base 74g for the performance of an immunoassay.

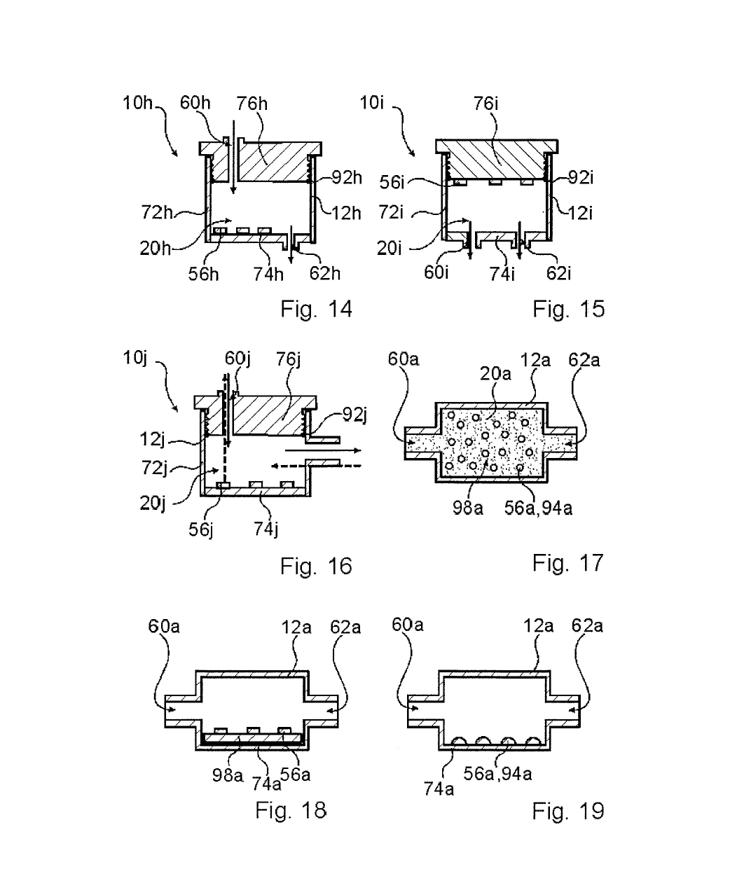

A further alternative reaction container 12h (FIG. 14) of an alternative device 10h has a lid 76h which is screwed into a base body 72h and which has an interface 60h for fluid introduction into a work volume 20h and is sealed with an O-ring 92h. A base 74h has capture antibodies 56h bound thereto and an interface 62h for liquid discharge arranged thereon. Under conditions of reduced gravity, liquids can be introduced and discharged in any desired directions. In a further alternative device 10i (FIG. 15), a reaction container 12i having a work volume 20i has a lid 76i which is screwed into a base body 72i and sealed with an O-ring 92i. Capture antibodies 56i are arranged bound to the lid 76i. Alternatively, instead of capture antibodies 56i, it is also possible for detection antibodies or other required analysis materials to be arranged bound to the lid 76i. Interfaces 60i, 62i are arranged on a base 74i.

In a further reaction container 12j (FIG. 16) having an alternative arrangement of interfaces 60j, 62j, the interface 60j for introduction of liquid volumes into a work volume 20j is arranged in a lid 76j, which is screwed into a base body 72j and sealed with an O-ring 92j. Capture antibodies 56j are arranged bound to a base 74j. Alternatively, instead of capture antibodies 56j, it is also possible for detection antibodies or other required analysis materials to be arranged bound to the base 74j. The interface 62j, which is intended for discharge of liquid volumes, is arranged on a lateral region of the base body 72j.

FIG. 17 shows alternative storage of the capture antibodies 56a in the reaction container 12a. The capture antibodies 56a are arranged bound to magnetic carrier bodies 94a and are arranged free-floating therewith in a suspension 98a in the work volume 20a. The magnetic carrier bodies 94a are intended to be moved by the magnet unit 110a. Alternatively, instead of capture antibodies 56a, it is also possible for detection antibodies 54a or other required analysis materials to be arranged bound to the magnetic carrier bodies 94a.

In further alternative storage (FIG. 18) of the capture antibodies 56a, they are arranged bound to a support 96a which is composed of plastic or glass or another transparent material and which is laid on the base 74a. Alternatively, however, it is also possible for the support 96a to be welded or adhesively bonded onto the base 74a or attached to a lid 76a. In a further alternative configuration, it is also possible for detection antibodies 54a, instead of capture antibodies 56a, to be stored in the aforementioned manner.

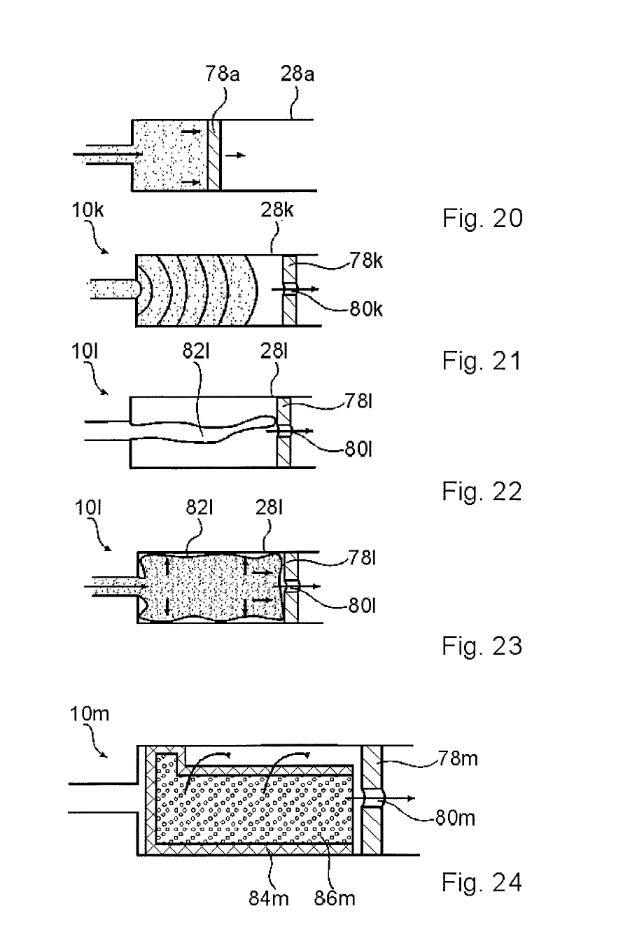

In further alternative storage (FIG. 19) of the capture antibodies 56a, they are arranged bound to magnetic carrier bodies 94a on a base 74a of the reaction container 12a in a dried state. As a result of supply of a liquid volume through one of the interfaces 60a, 62a, they are brought into solution and are then intended for mixing by means of the magnet unit 110a. In a further alternative configuration, it is also possible for detection antibodies 54a, instead of or in addition to the capture antibodies 56a, to be stored in the aforementioned manner. Similarly, it is possible, as an alternative, to store capture antibodies 56a and detection antibodies 54a as a mix on the same surface. In a further alternative configuration, capture antibodies 56a on the base 74a or on the lid 76a of the reaction container 12a and detection antibodies 54a on the lid 76a or on the base 74a of the reaction container 12a, bound in each case to magnetic carrier bodies 94a, can be stored separately from one another in a dried state.

FIG. 20 shows the waste container 28a of the device 10a during a filling operation. The waste container 28a has a movable plunger 78a which is withdrawn for filling or pushed back during filling.

In an alternative waste container 28k (FIG. 21) of an alternative device 10k, a movable plunger 78k has a filter 80k which is intended for pressure equalization during filling. By means of the filter 80k, the waste container 28k is designed for pressure-equalization operation. The filter 80k is implemented as a hydrophobic filter; depending on the medium used in an analysis, the filter 80k can also be implemented as a hydrophilic filter.

A further alternative device 101 has an inserted collection container 82l (FIG. 22) which expands during filling (FIG. 23). For pressure equalization during filling, a movable plunger 78l has a hydrophobic filter 80l. By means of the hydrophobic filter 80l, the waste container 28l is designed for pressure-equalization operation.

An alternative waste container 28m (FIG. 24) of a device 10m has a wicking body 84m filled with absorbent material 86m. During filling of the waste container 28m, air is displaced from the wicking body 84m and excess liquid volume is bound by the absorbent material 86m. The absorbent material 86m can, for example, be formed by organic absorbents such as nondrip organic sponge material, by capillary plastic storers, as produced by the firm POREX for example, by hygroscopic materials such as, for example, silica gel or organic superabsorbent materials such as, for example, the product sold by BASF under the trade name Luquasorb®. Instead of the absorbent material 86m, it is also possible to use an adsorbent material, for example sintered plastic storers or mineral adsorbents such as dried clay minerals or activated carbon. In alternative configurations, the wicking body 84m can be intended for taking in excess liquid volumes. The waste container 28k has a hydrophobic filter 80m in a movable plunger and is designed for pressure-equalization operation.

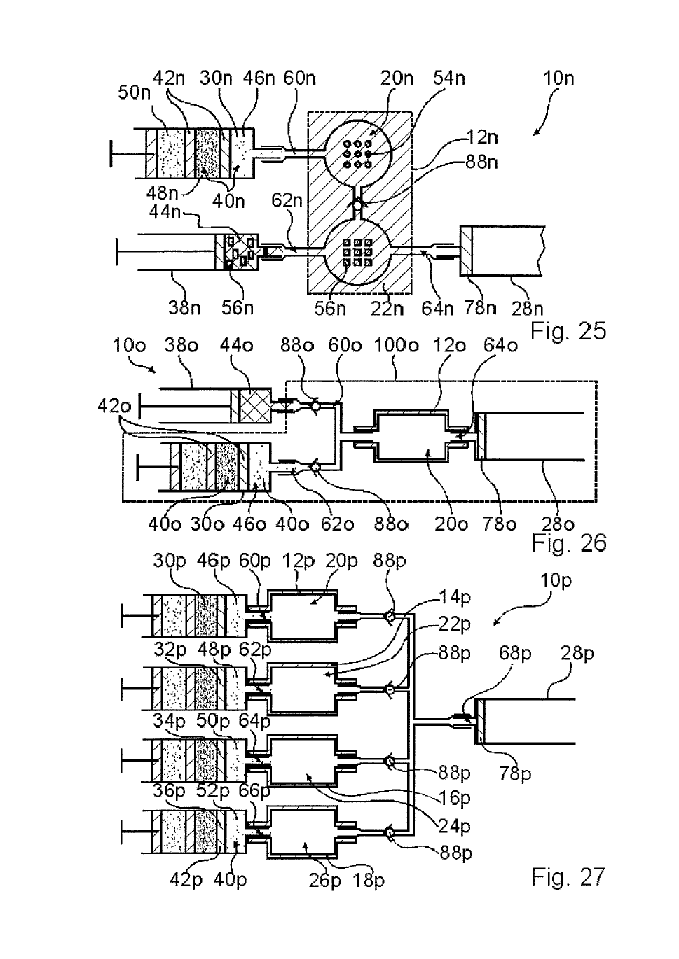

An alternative device 10n (FIG. 25) has a reaction container 12n having two work volumes 20n, 22n in which detection antibodies 54n and capture antibodies 56n, respectively, are bound, preferably in dried form, and which are connected via a connection to a valve 88n. The work volumes 20n, 22n are connected via interfaces 60n, 62n, 64n to further media containers implemented as a sample container 38n containing a sample 44n with magnetic mixing bodies 58n as a mix, of an analysis-material container 30n implemented as a multichamber syringe containing a plurality of analysis materials 46n, 48n, 50n in a plurality of subchambers 40n divided by separators 42n, and of a waste container 28n having a movable plunger 78n.

In an alternative device 10o (FIG. 26), a reaction container 12o, which a work volume 20o, an analysis-material container 30o and a waste container 28o are preassembled to form a module 100o which is intended for connection to a further media container implemented as a sample container 38o. The module 100o is connected to the sample container 38o containing a sample 44o via an interface 60o having a valve 88o. An interface 62o within the module 100o, which interface connects the work volume 20o to the analysis-material container 30o implemented as a multichamber syringe, likewise has a valve 88o.

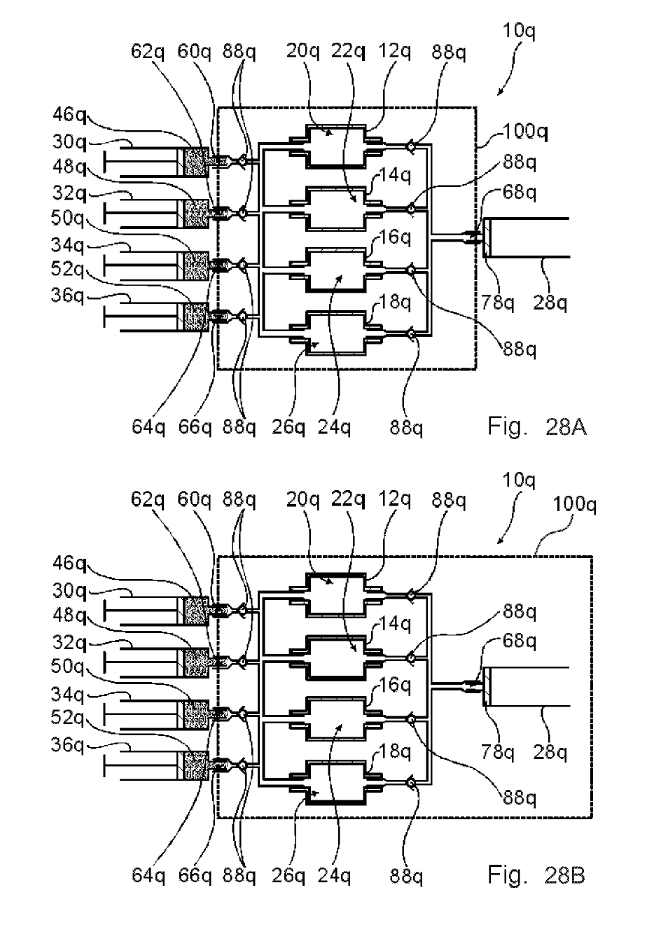

An alternative device 10p (FIG. 27) has four reaction containers 12p, 14p, 16p, 18p having work volumes 20p, 22p, 24p, 26p which are connected in each case via an interface 60p, 62p, 64p, 66p to analysis-material containers 30p, 32p, 34p, 36p implemented as multichamber syringes. The work volumes 20p, 22p, 24p, 26p are connected via a common interface 68p having valves 88p to a waste container 28p having a movable plunger 78p. The device 10p is intended for parallel performance of a plurality of biochemical analyses. In the device 10p, it is possible to perform in parallel a plurality of similar biochemical analyses, for example analysis of the same or different samples for the same analyte, and/or a plurality of different biochemical analyses, for example an analysis of a plurality of volumes of a sample for different analytes in each case.

In a further alternative device 10q (FIG. 28A), four reaction containers 12q, 14q, 16q, 18q arranged in parallel and having work volumes 20q, 22q, 24q, 26q are preassembled to form a module 100q which is intended to allow parallel performance of a plurality of biochemical analyses. The module 100q is connected via interfaces 60q, 62q, 64q, 66q, which have valves 88q, to analysis-material containers 30q, 32q, 34q, 36q in which an analysis material 46q, 48q, 50q, 52q is stored in each case. The work volumes 20q, 22q, 24q, 26q are connected to a waste container 28q having a movable plunger 78q via a common interface 68q having valves 88q. In alternative configurations, the module 100q can also additionally comprise the waste container 28q (FIG. 28B) and/or one or more of the analysis-material containers 30q, 32q, 34q, 36q.

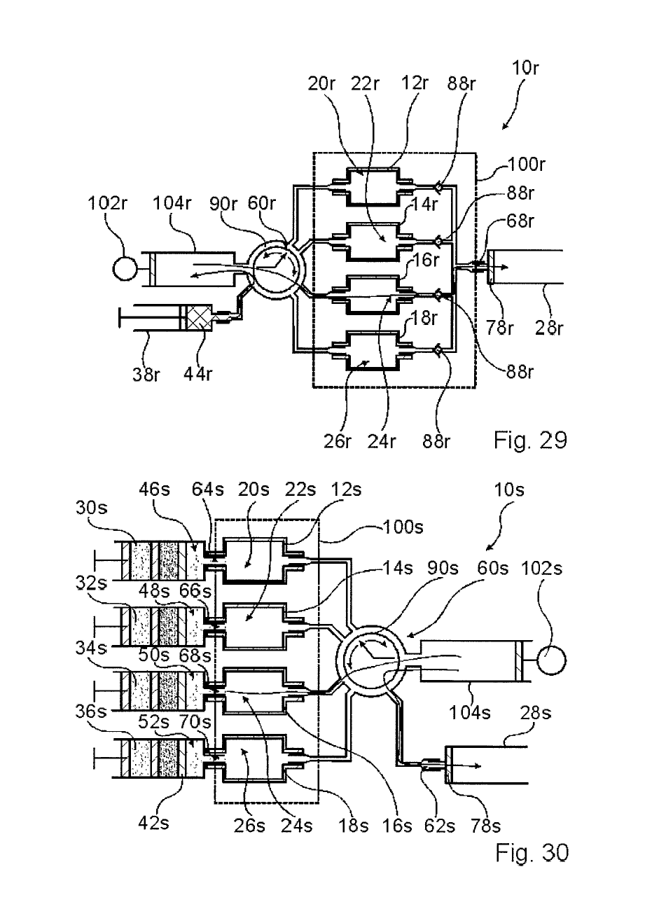

In a further alternative device 10r (FIG. 29), four reaction containers 12r, 14r, 16r, 18r arranged in parallel and having work volumes 20r, 22r, 24r, 26r are likewise preassembled to form a module 100r which is intended to allow sequential or partially parallel performance of a plurality of biochemical analyses. The reaction containers 12r, 14r, 16r, 18r are charged via an interface 60r which has a multiport valve 90r. The multiport valve 90r is designed to transfer a sample 44r from a sample container 38r into a motorized syringe 104r which is coupled to a motor 102r. By means of the motor 102r, the sample 44r is released from the motorized syringe 104r by means of elevated pressure and sent to one of the work volumes 20r, 22r, 24r, 26r using the multiport valve 90r. In principle, it is possible for the multiport valve 90r to be connected to further sample containers 38r or to further media containers. For continuation of the biochemical analysis, the sample container 38r is replaced by a media container containing further materials for the biochemical analysis, which materials are likewise released via the multiport valve 90r and the motorized syringe 104r. Alternatively, the sample container 38r can be implemented as a multichamber syringe and store further reagents for the biochemical analysis. An interface 62r connects the module 100r to a waste container 28r which, in alternative developments, can also be included in the module 100r.

In a further alternative device 10s (FIG. 30), four reaction containers 12s, 14s, 16s, 18s arranged in parallel and having work volumes 20s, 22s, 24s, 26s are likewise preassembled to form a module 100s which is intended to allow sequential or partially parallel performance of a plurality of biochemical analyses. The work volumes 20s, 22s, 24s, 26s are connected via interfaces 64s, 66s, 68s, 70s to analysis-material containers 30s, 32s, 34s, 36s implemented as multichamber syringes having subchambers 40s partitioned off by separators 42s. An interface 60s common to the four work volumes 20s, 22s, 24s, 26s has a multiport valve 90s which is connected to a motorized syringe 104s which is coupled to a motor 102s. Via the multiport valve 90s and the motorized syringe 104s, liquid volumes are sucked from media containers by means of reduced pressure and introduced specifically into individual work volumes 20s, 22s, 24s, 26s. Excess liquid volumes are sucked specifically from the work volumes 20s, 22s, 24s, 26s and transferred into the motorized syringe 104s. By means of the motor 102s, the excess liquid volume is then ejected from the motorized syringe 104r under elevated pressure and sent to a waste container 28s having a movable plunger 78s using the multiport valve 90r. In alternative configurations, the waste container 28s can also be included in the module 100s.

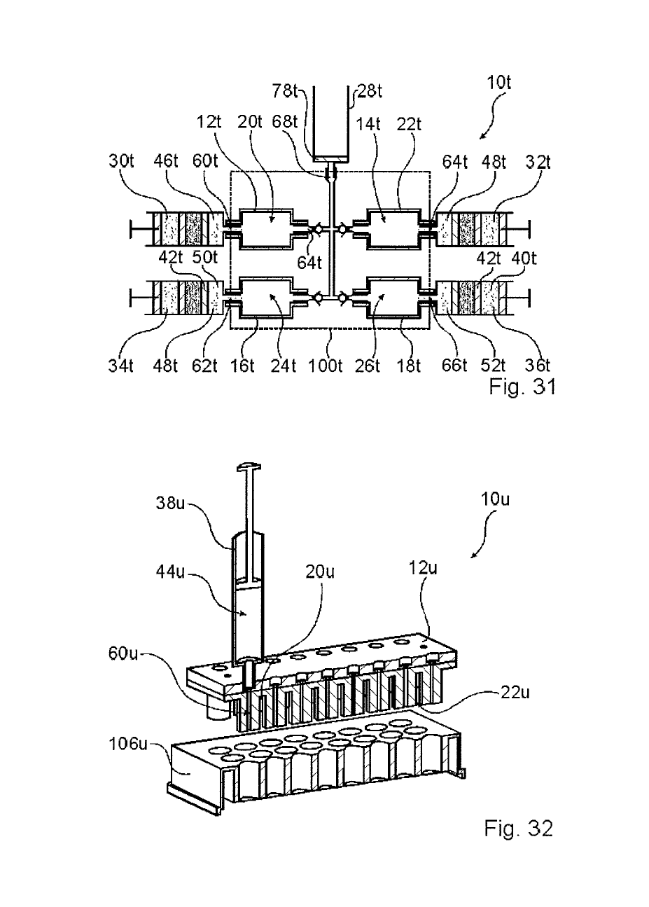

In a further alternative device 10t (FIG. 31), four reaction containers 12t, 14t, 16t, 18t having work volumes 20t, 22t, 24t, 26t in a two-rowed arrangement are preassembled to form a module 100t which is intended to allow parallel performance of a plurality of biochemical analyses. The work volumes 20t, 22t, 24t, 26t are connected to a common waste container 28t having a movable plunger 78t via a common interface 68t having valves 88t. The work volumes 20t, 22t, 24t, 26t are connected to analysis-material containers 30t, 32t, 34t, 36t implemented as multichamber syringes via interfaces 60t, 62t, 64t, 66t. In alternative configurations, the waste container 28t and/or one or more of the analysis-material containers 30t, 32t, 34t, 36t can be preassembled in the module 100t.

FIG. 32 shows a reaction container 12u of an alternative device 10u, which container is intended as a connection block for connection to a commercial multiwell plate 106u, and which container has a multiplicity of work volumes 20u, 22u (for the sake of clarity, further work volumes have been left unidentified). As a result of the connection, individual wells of the multiwell plate 106u are used as base elements of the work volumes 20u, 22u and complete the reaction container 12u to form a substantially completely closed vessel. Interfaces 60u connect the work volumes 20u, 22u to further media containers such as a sample container 38u, which stores a sample 44u, or a waste container (not shown here).

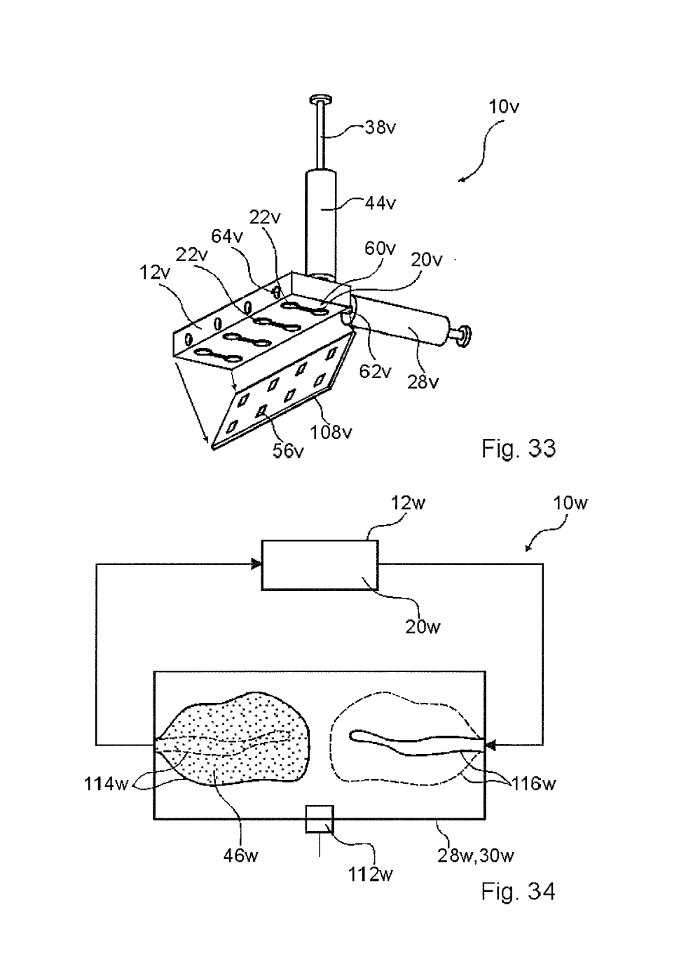

FIG. 33 shows a reaction container 12v of an alternative device 10v having a multiplicity of work volumes 20v, 22v, which container is assembled with a commercial planar array 108v containing capture antibodies 56v bound thereto as spots to form a substantially completely closed vessel. Assembly can be achieved via interlocking and/or force-fitting, for example by adhesive bonding or welding. Interfaces 60v, 62v, 64v are intended for connection of the work volumes 20v, 22v to sample containers 38v, which store a sample 44v, to waste containers 28v and/or to further media containers.

FIG. 34 shows an alternative device 10w having a reaction container 12w which has a work volume 20w, and having an analysis-material container 30w which is integrated with a waste container 28w. The analysis-material container 30w integrated with a waste container 28w has a compartment 114w for taking in analysis materials 46w and a compartment 116w for taking in excess liquid volumes; both are in the form of elastic uptake sacks, with the compartment 116w for taking in excess liquid volumes being empty and folded up prior to the start of an analysis reaction. Owing to emptying of the compartment 114w for the analysis materials 46w over the course of performance of a biochemical analysis, the compartment for taking in excess liquid volumes 116w can expand when filling up. A dashed line is used to show a state of the analysis-material container 30w integrated with a waste container 28w after performance of the analysis, with emptied compartment 114w for the analysis materials 46w and filled compartment 116w for taking in excess liquid volumes. Volume-neutral storage is attained. The analysis-material container 30w has a valve 112w for the purposes of venting, in order to achieve pressure-neutral operation. Emptying of the compartment 114w for the analysis materials 46w is achieved by suction; in alternative configurations, emptying can, for example, be achieved by a movable plunger which exerts pressure on the compartment 114w for the analysis materials 46w. In alternative configurations, the compartments 114w, 116w can, for example, have movable closure elements for alteration of their volumes or fixed volumes, instead of being in the form of elastic uptake sacks.

1. A device for performing a biochemical analysis, especially in outer space, more particularly an immunoassay, in which analysis at least one analyte in a sample is determined selectively, having at least one reaction container which has at least one work volume intended for taking in a liquid volume and for performing at least one substep of an analysis reaction, and having at least one interface intended for connecting at least one work volume to a further media container,
wherein
the reaction container is implemented as a container which is at least substantially completely closed in an assembled state.

2. The device according to claim 1, wherein the work volume is designed for reaction performance under conditions of reduced gravity.

3. The device according to claim 2, wherein the work volume has a shape which widens starting from an interface.

4. The device according to claim 3, wherein the work volume is at least substantially rectangular.

5. The device according to claim 3, wherein the work volume is in a drop shape.

6. The device according to claim 3, wherein the work volume is in a circular shape.

7. The device according to claim 3, wherein the work volume is in a nozzle shape.

8. The device according to claim 1, further comprising at least one additional media container implemented as a waste container intended for taking in excess liquid volumes.

9. The device according to claim 8, wherein the waste container is at least substantially completely closed.

10. The device according to claim 8, wherein the waste container is designed for pressure-equalization operation.

11. The device at least according to claim 8, wherein the waste container has at least one wicking body.

12. The device according to claim 1, further comprising at least one additional media container implemented as an analysis-material container intended for providing analysis materials.

13. The device according to claim 12, wherein the analysis-material container is implemented as a multichamber syringe.

14. The device according to claim 12, wherein the analysis-material container is integrated with a waste container.

15. The device according to claim 1, wherein at least one reaction container is preassembled together with at least one further reaction container and/or at least one further media container to form a module intended for connection to a further media container.

16. The device according to claim 15, wherein the module is intended for allowing parallel performance of a plurality of biochemical analyses.

17. The device according to claim 1, further comprising magnetic mixing bodies intended for mixing reaction materials and the sample for an analysis reaction.

18. A method for performing a biochemical analysis using a device according to claim 1.

19. The method according to claim 18, wherein the performance is carried out under conditions of reduced gravity.

20. The method according to claim 18, wherein mixing of analysis materials and the sample for the analysis reaction is carried out by means of magnetic mixing bodies.

21. The method at least according to claim 18, wherein only reaction materials and samples within a work volume of a reaction container are involved in an analysis reaction.

22. The method at least according to claim 18, wherein addition of reaction materials and samples can proceed in any small subvolumes and with pauses included.
